(19) United States
(12) Patent Application Publication
(10) Pub. No.: US 2005/0048652 A1
(43) Pub. Date: Mar. 3, 2005

(54) Retroelement vector system for amplification and delivery of nucleotide sequences in plants

(75) Inventors: Daniel Voytas, Ames, IA (US); David Wright, Ames, IA (US)

Correspondence Address: MCKEE, VOORHEES & SEASE, P.L.C., 801 GRAND AVENUE, SUITE 3200, DES MOINES, IA, 50309-2721, US

(73) Assignee: Iowa State University Research Foundation, Inc., Ames, IA (US)

(21) Appl. No.: 10/920,768

(22) Filed: Aug. 18, 2004

Related U.S. Application Data
(60) Provisional application No. 60/496,319, filed on Aug. 19, 2003.

(51) Int. Cl. C12Q 1/68; A01H 1/00; C12N 15/87; C12N 5/04

(52) U.S. Cl. 435/468; 800/278; 435/419

(57) ABSTRACT

A novel mini-retrotransposon vector system is provided for integrating foreign DNA into plants. The invention includes a novel vector comprising a truncated and modified retroelement which includes the 5′ and 3′ LTR regions that provide transcription initiation and termination sites as well as the cis acting sequences required for reverse transcription. Novel vectors, plant cells, and methods of using the same are disclosed.

### CROSS-REFERENCE TO RELATES APPLICATIONS

This application is a conversion of U.S. Provisional Application No. 60/496,319, filed Aug. 19, 2003, which is herein incorporated by reference in its entirety.

### GRANT REFERENCE

This invention was supported at least in part by DOE Contract No. DE-FC05-920R22072 The United States government may have certain rights in this invention.

### BACKGROUND OF THE INVENTION

Retrotransposons are mobile genetic elements that replicate through reverse transcription of an mRNA intermediate. There are two major classes of retrotransposons, which are distinguished by whether or not they are flanked by long terminal direct repeats (LTRs). The LTR-containing retrotransposons are structurally similar to the retroviruses. Although the LTRs are identical in sequence, they serve different functions. The 5′ LTR acts as the promoter, whereas the 3′ LTR provides the polyadenylation signal and the polyadenylation site. Between the LTRs are a Primer Binding Site (PBS), PolyPurine Tract (PPT) and the mRNA packaging signal. The PBS and PPT act as primer sites for the initiation of DNA synthesis, and the packaging signal ensures that the viral RNA is taken into the particle.

Both the LTR-retrotransposons and retroviruses (collectively referred to as retroelements) undergo a similar replication cycle, with the primary difference being that the retrotransposons are not infectious. The life cycle begins with the synthesis of an element mRNA, which is translated to yield the protein products required for replication and also serves as a template for reverse transcription. Retroelements encode a Gag polyprotein that assembles into a virus or virus-like particle in the cell cytoplasm. Packaged within this particle are template mRNA and the Pol gene products, among which is reverse transcriptase. Upon completion of DNA synthesis through reverse transcription, the cDNA is carried into the nucleus by the integration complex, a key component of which is the pol-encoded integrase protein. Integrase carries out the cutting and joining steps of integration and is necessary for high efficiency integration in vivo.

Retroelements have been harnessed for use as gene delivery vectors (Miller, 1997). Retroelement-based gene delivery strategies typically utilize a two-component vector system. The first component is a vector retroelement in which all of the retroelement genes have been replaced with a gene(s) of interest. Upon integration into the host cell, the vector retroelement is not capable of additional rounds of replication, and the gene of interest is maintained and expressed in the host cell. The second component of the vector system is a helper retroelement. Helper retroelements express all proteins required for replication. When a cell is expressing both a helper and vector retroelement, functional replication intermediates are formed. In the case of the retroviruses, these replication intermediates are virus particles that can be harvested and used to infect the tissue of choice to deliver the target gene. For the retrotransposons, the replication intermediates are virus-like particles that carry a copy of the vector retroelement mRNA. This mRNA is copied into cDNA, which can then integrate into the host genome.

Two-component retroviral vector systems are used for gene delivery in human gene therapy (Miller, 1997). Two-component retrotransposon gene delivery systems have also been developed, but these systems have been limited to lower eukaryotes, specifically yeasts (Boeke et al., 1988; Levin and Boeke, 1992; Zou et al., 1996). In this application, we describe the development of a two-component retroelement gene delivery system.

### BRIEF SUMMARY OF THE INVENTION

According to the invention a mini-retrotransposon vector system has been established for integrating foreign DNA into plants. The invention includes a novel vector comprising a truncated and modified retroelement which includes the 5′ and 3′ LTR regions including the cis acting sequences required for reverse transcription (5′ LTR with or without a +1 fusion to the CaMV 35S promoter, a primer binding site, a portion of gag, a polylinker with unique restriction sites to facilitate cloning of exogenous DNA, a polypurine tract and the 3′ LTR) SEQ ID NO:1 and 2. According to the invention, any retrotransposon may be used to generate the mini vectors of the invention. This includes but is not limited to Tto1 from tobacco, (Hirochika, 1993), Tnt1 from tobacco (Pouteau et al, 1991, or Tos17 from rice (Hirochika et al 1996). These retrotransposons all have homologues in various species which may be used according to the invention. The vector system according to the invention provides for integration through the use of two complete long terminal repeat regions of a retrotransposon which provide transcription initiation and termination sites. Also included are the cis acting sequences required for reverse transcription, namely the PBS and PPT. The exogenous DNA is placed through the use of a multiple cloning site into the vector between the LTR regions. In a preferred embodiment the 5′ LTR is operably linked to a promoter more preferably a constitutive promoter to provide for autonomous replication of the sequences for high copy number.

Also disclosed is a method for introducing heterologous nucleotide sequences into plant chromosomes. According to the method the mini-retrotransposon vector is then introduced to a cell using standard molecular biology techniques known of those of skill in the art. The vector must be introduced in the presence of the remainder of the retroelement including gag and pol. These may be provided in the form of a helper vector, which is either previously integrated or co-transformed or, optionally may be provided by host native retroelement sequences as long as the host sequences are compatible with the mini-transposon vector sequences. In the presence of helper retroelements, reverse transcribed cDNA is integrated into the plant chromosome through the action of retroelement encoded integrase.

Any plant cell may be used in accordance with the invention, and the invention includes vectors, helper cell lines, helper vectors, as well as modified plant cells, and plants which incorporate exogenous integrated DNA. Incorporation of exogenous DNA into plant chromosomes provides for a heritable genotype that is desired for any of a number of purposes known to those of skill in the art, to, for example, provide pest resistance, herbicide resistance, alter fatty acid metabolism, and improve reaction to stress and the like.

### DETAILED DESCRIPTION OF THE FIGURES

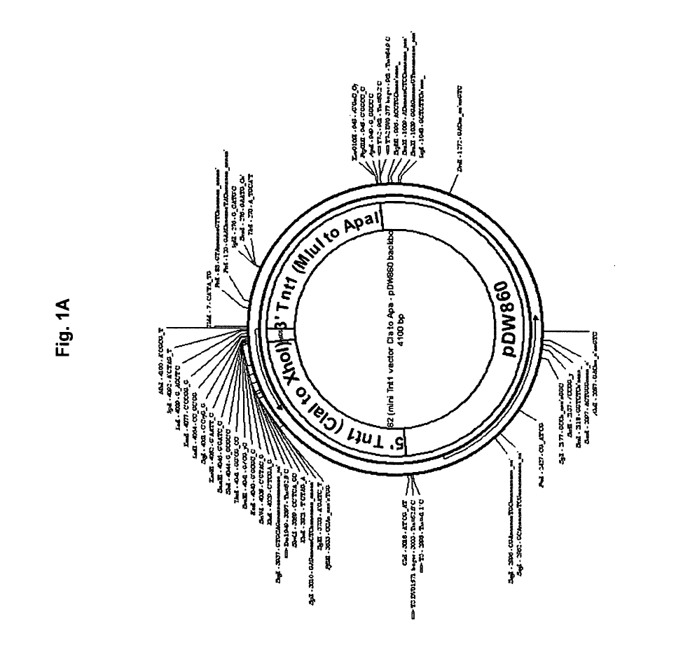

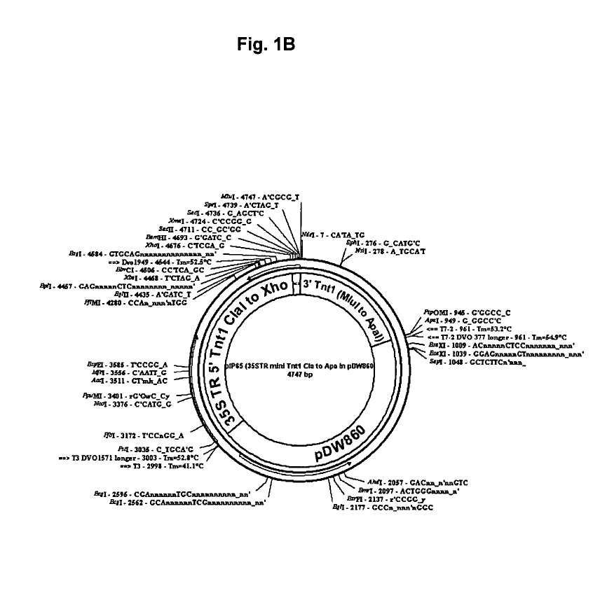

FIG. 1A; is a map of the first mini retroelement vector according to the invention (expression of which will be induced under stress).

FIG. 1B is a map of the second vector (under the control of the CaMV 35S promoter, constitutive expression of the mini-element).A poly linker with unique restriction sites was added to each of the vector retroelements to facilitate cloning of exogenous DNA.

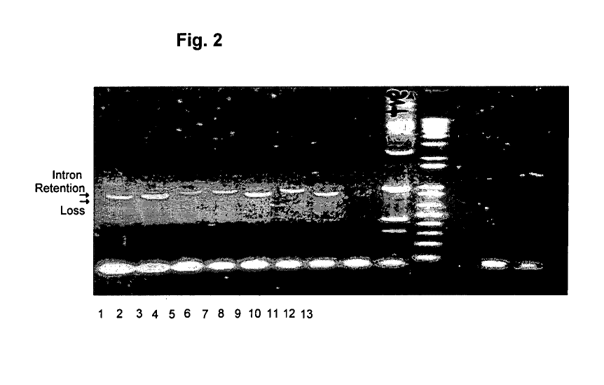

FIG. 2 show the PCR results for intron loss from Tnt1 mini retroelement vector. Lanes 1, 2, 5, 7 indicate intron loss through reverse transcription. Lanes 3, 4,and 6 indicate intron retention. Lane 8 indicates two mini element vectors one with and one without an intron. Lane 9 is a positive control using the mini element on a cloning vector. Lane 10 is a DNA marker. Lane 11 is empty. Lane 12 and 13 are negative controls.i.e. cDNA is about 80 bp shorter than the parental construct.

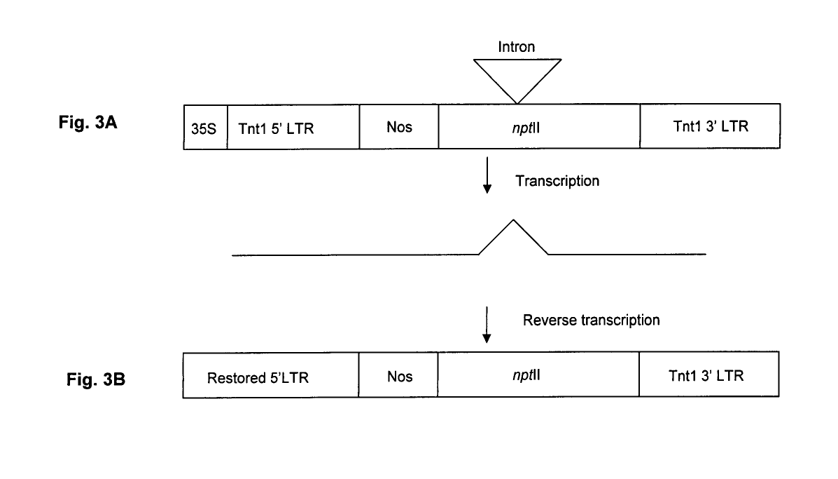

FIG. 3A depicts a pictorial summary PCR assay for replication by reverse transcription

FIG. 3B show the results depicting the restoration of the 5′ end of the LTR that was observed in three of twenty calli analyzed.

### DETAILED DESCRIPTION OF THE INVENTION

Retroelement-based gene delivery strategies typically utilize a two-component vector system. The first component is a vector retroelement in which all of the retroelement genes have been replaced with a gene(s) of interest. Upon integration into the host cell, the vector retroelement is typically not capable of additional rounds of replication, and the gene of interest is maintained and expressed in the host cell. The second component of the vector system is a helper retroelement. Helper retroelements express all proteins required for replication. When a cell is expressing both a helper and vector retroelement, functional replication intermediates are formed. In the case of the retroviruses, these replication intermediates are virus particles that can be harvested and used to infect the tissue of choice to deliver the target gene. For the retrotransposons, the replication intermediates are virus-like particles that carry a copy of the vector retroelement mRNA. This mRNA is copied into cDNA, which can then integrate into the host genome.

Two-component retroviral vector systems are used for gene delivery in human gene therapy (Miller, 1997). Two-component retrotransposon gene delivery systems have also been developed, but these systems have been limited to lower eukaryotes, specifically yeasts (Boeke et al., 1988; Levin and Boeke, 1992; Zou et al., 1996).

The present invention provides nucleic acids, as well as vectors, cells, and plants (including plant parts, seeds, and embryos) containing the nucleic acids. In particular, molecular tools are provided in the form of nucleic acids that are retroelements or that contain retroelement sequences. The invention also features methods for manipulating such nucleic acids. For example, the invention features methods to introduce nucleic acids containing retroelements or retroelement sequences into cells, especially retroelements carrying at least one agronomically significant characteristic. Specifically, the invention provides a method to transfer agronomically significant characteristics to plants, in which a helper cell line that expresses gag and pol sequences is used to enable transfer of the secondary construct that carries an agronomically significant characteristic and has retroelement sequences that allow for replication and integration.

According to the invention a mini-retrotransposon vector system has been established for integrating foreign DNA into plants. The invention includes a novel vector comprising a truncated and modified retroelement which includes the 5′ and 3′ LTR regions including the cis acting sequences required for reverse transcription (5′ LTR with or without a +1 fusion to the CaMV 35S promoter, a primer binding site, a portion of gag, a polylinker with unique restriction sites to facilitate cloning of exogenous DNA, a polypurine tract and the 3′ LTR) SEQ ID NO:1 and 2. According to the invention, any retrotransposon may be used to generate the mini vectors of the invention, this includes but is not limited to Tto1 from tobacco, (Hirochika, 1993), Tnt1 from tobacco (Pouteau et al, 1991, or Tos17 from rice (Hirochika et al 1996). These retrotransposons all have homologues from various species, which may be used according to the invention. The vector system according to the invention provides for integration through the use of two complete long terminal repeat regions of a retrotransposon which provide transcription initiation and termination sites as well as the cis acting sequences required for reverse transcription. The exogenous DNA is placed through the use of a multiple cloning site into the vector between the LTR regions. In a preferred embodiment the 5′ LTR is operably linked to a promoter more preferably a constitutive promoter to provide for autonomous replication of the sequences for high copy number.

Also disclosed is a method for introducing heterologous nucleotide sequences into plant chromosomes. According to the method the mini-retrotransposon vector is then introduced to a cell using standard molecular biology techniques known of those of skill in the art. The vector must be introduced in the presence of the remainder of the retroelement including gag and pol. These may be provided in the form of a helper vector, which is either previously integrated or co-transformed or, optionally may be provided by host native retroelement sequences as long as the host sequences are compatible with the mini-transposon vector sequences. Compatibility may be easily ascertained using no more than routine experimentation and the teaching and assays disclosed herein. In a preferred embodiment the mini-vector is composed of Tnt1 elements and the gag and pol genes are provided due to activation of host retroelements by microbial elicitors, pathogens and abiotic stresses such as wounding and/or freezing. In the presence of helper retroelements, transcribed cDNA is integrated into the plant chromosome through the action of retroelement encoded integrase.

Any plant cell may be used in accordance with the invention, and the invention includes vectors, helper cell lines, helper vectors, as well as modified plant cells, and plants which incorporate exogenous integrated DNA. Incorporation of exogenous DNA into plant chromosomes provides for a heritable genotype that is desired for any of a number of purposes known to those of skill in the art, to, for example, provide pest resistance, herbicide resistance, alter fatty acid metabolism, and improve reaction to stress and the like.

For purposes of this application the following terms shall have the definitions recited herein. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5^{th} edition, 1993). The terms defined below are more fully defined by reference to the specification as a whole.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Canteen, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Washington, D.C. (1993). The product of amplification is termed an amplicon.

As used herein, “antisense orientation” includes reference to a duplex polynucleotide sequence that is operably linked to a promoter in an orientation where the antisense strand is transcribed. The antisense strand is sufficiently complementary to an endogenous transcription product such that translation of the endogenous transcription product is often inhibited.

As used herein, “chromosomal region” includes reference to a length of a chromosome that may be measured by reference to the linear segment of DNA that it comprises. The chromosomal region can be defined by reference to two unique DNA sequences, i.e., markers.

The term “conservatively modified variants” applies to both amino acid and nucleic acid sequences. With respect to particular nucleic acid sequences, conservatively modified variants refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W). See also, Creighton (1984) Proteins W.H. Freeman and Company.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al. Nucl. Acids Res. 17:477-498 (1989)). Thus, the maize preferred codon for a particular amino acid may be derived from known gene sequences from maize. Maize codon usage for 28 genes from maize plants are listed in Table 4 of Murray et al., supra.

As used herein “full-length sequence” in reference to a specified polynucleotide or its encoded protein means having the entire amino acid sequence of, a native (non-synthetic), endogenous, biologically active form of the specified protein. Methods to determine whether a sequence is full-length are well known in the art including such exemplary techniques as northern or western blots, primer extensions, S1 protection, and ribonuclease protection. See, e.g., Plant Molecular Biology: A Laboratory Manual, Clark, Ed., Springer-Verlag, Berlin (1997). Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

By “host cell” is meant a cell which contains a vector and supports the replication and/or expression of the vector. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is: (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material can be performed on the material within or removed from its natural state. For example, a naturally occurring nucleic acid becomes an isolated nucleic acid if it is altered, or if it is transcribed from DNA which has been altered, by means of human intervention performed within the cell from which it originates. See, e.g., Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (e.g., a promoter) becomes isolated if it is introduced by non-naturally occurring means to a locus of the genome not native to that nucleic acid. Nucleic acids which are “isolated” as defined herein, are also referred to as “heterologous” nucleic acids.

As used herein, “localized within the chromosomal region defined by and including” with respect to particular markers includes reference to a contiguous length of a chromosome delimited by and including the stated markers.

As used herein, “marker” includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes of that pair to be followed. A genotype may be defined by use of one or a plurality of markers.

As used herein, “nucleic acid” or “nucleotide” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., Molecular Cloning—A Laboratory Manual, 2^{nd} ed., Vol. 1-3 (1989); and Current Protocols in Molecular Biology, F. M. Ausubel et al., Eds., Current Protocols, a joint venture between Greene Publishing Associates, Inc. and John Wiley & Sons, Inc. (1994).

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “phenotype” includes the morphology, physiology, biochemistry, or gene expression alterations in any of the above from that of the untransformed plant.

As used herein, the term “plant” can include reference to whole plants, plant parts or organs (e.g., leaves, stems, roots, etc.), plant cells, seeds and progeny of same. Plant cell, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. The class of plants which can be used in the methods of the invention is generally as broad as the class of higher plants amenable to transformation techniques, including both monocotyledonous and dicotyledonous plants. Particularly preferred plants include maize, soybean, sunflower, sorghum, canola, wheat, alfalfa, cotton, rice, barley, and millet.

As used herein, “polynucleotide” includes reference to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons as “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides are not entirely linear. For instance, polypeptides may be branched as a result of ubiquitination, and they may be circular, with or without branching, generally as a result of posttranslation events, including natural processing event and events brought about by human manipulation which do not occur naturally. Circular, branched and branched circular polypeptides may be synthesized by non-translation natural process and by entirely synthetic methods, as well. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. The following is a list of tissue preferred or tissue specific promoters.

A “cell type” specific promoter primarily drives expression in certain cell types in one or more organs, for example, vascular cells in roots or leaves. An “inducible” or “repressible” promoter is a promoter which is under environmental control. Examples of environmental conditions that may effect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, preferably 90% sequence identity, and most preferably 100% sequence identity (i.e., complementary) with each other.

The term “stringent conditions” or “stringent hybridization conditions” includes reference to conditions under which a probe will hybridize to its target sequence, to a detectably greater degree than to other sequences (e.g., at least 2-fold over background). Stringent conditions are sequence-dependent and may be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaC1, 1% SDS (sodium dodecyl sulphate) at 37° C., and a wash in 1× to 2× SSC (20×SSC=3.0 M NaC1/0.3 M trisodium citrate) at 50 to 55° C. Exemplary moderate stringency conditions include hybridization in 40 to 45% formamide, 1 M NaC1, 1% SDS at 37° C., and a wash in 0.5× to 1× SSC at 55 to 50° C. Exemplary high stringency conditions include hybridization in 50% formamide, 1 M NaC1, 1% SDS at 37° C., and a wash in 0.1× SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6(log M)+0.41(% GC)−0.61(% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acids Probes, Part I, Chapter 2, Ausubel, et al., Eds., Greene Publishing and Wiley-Interscience, New York (1995).

As used herein, the term “structural gene” includes any nucleotide sequence the expression of which is desired in a plant cell. A structural gene can include an entire sequence encoding a protein, or any portion thereof. Examples of structural genes are included hereinafter are intended for illustration and not limitation.

As used herein, “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et a., Nucleic Acids Res. 25:3389-3402 (1997). Software for performing BLAST analyses is publicly available, e.g., through the National Center for Biotechnology-Information Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity”or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e)(I) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

(e)(ii) The terms “substantial Identity” in the context of a peptide indicates that a peptide comprises a sequence with at least 70% sequence identity to a reference sequence, preferably 80%, or preferably 85%, most preferably at least 90% or 95% sequence identity to the reference sequence over a specified comparison window. Optionally, optimal alignment is conducted using the homology alignment algorithm of Needleman and Wunsch, J. Mol. Biol. 48:443 (1970). an indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions which are not identical may differ by conservative amino acid changes.

The present invention provides nucleic acids, as well as vectors, cells, and plants (including plant parts, seeds, and embryos) containing the nucleic acids. In particular, molecular tools are provided in the form of nucleic acids that are retroelements or that contain retroelement sequences. The invention also features methods for manipulating such nucleic acids. For example, the invention features methods to introduce nucleic acids containing retroelements or retroelement sequences into cells, especially retroelements carrying at least one agronomically significant characteristic. Specifically, the invention provides a method to transfer agronomically significant characteristics to plants, in which a helper cell line that expresses gag and pol sequences is used to enable transfer of the secondary construct that carries an agronomically significant characteristic and has retroelement sequences that allow for replication and integration.

Nucleic acid molecules of the invention also can contain at least one nucleic acid sequence that imparts an agronomically significant characteristic. Agronomically significant characteristics can include, without limitation, those selected from the group consisting of: male sterility, self-incompatibility, foreign organism resistance, improved biosynthetic pathways, environmental tolerance, photosynthetic pathways, nutrient content, fruit ripening, oil biosynthesis, pigment biosynthesis, seed formation, starch metabolism, salt tolerance, cold/frost tolerance, drought tolerance, tolerance to anaerobic conditions, protein content, carbohydrate content (e.g., sugar and starch content), amino acid content, and fatty acid content.

In another aspect, the invention features seeds and plants containing the nucleic acid molecules provided herein. Suitable plants include, for example, soybean, maize, sugar cane, beet, tobacco, wheat, barley, poppy, rape, sunflower, alfalfa, sorghum, rose, carnation, gerbera, carrot, tomato, lettuce, chicory, pepper, melon, cabbage, oat, rye, cotton, flax, potato, pine, walnut, citrus fruits, hemp, oak, rice, petunia, orchids, Arabidopsis, broccoli, cauliflower, Brussels sprouts, onion, garlic, leek, squash, pumpkin, celery, peas, beans, strawberries, grapes, apples, pears, peaches, banana, palm, cocoa, cucumber, pineapple, apricot, plum, sugar beet, lawn grasses, maple, triticale, safflower, peanut, and olive.

Recombination constructs can be made using the starting materials above or with additional materials, using methods well-known in the art. In general, the sequences can be manipulated to have ligase-compatible ends, and incubated with ligase to generate full constructs. For example, restriction enzymes can be chosen on the basis of their ability to cut at an acceptable site in both sequence to be ligated, or a linker can be added to adapt the sequence end(s) to be compatible. The methods for conducting these types of molecular manipulations are well known in the art, and are described in detail in Sambrook et al., supra; and Ausubel et al. (1993) Current Protocols in Molecular Biology (Greene Publishing Associates, Inc.). The methods described herein according to Tinland et al. (1994) Proc. Natl. Acad. Sci. USA 91:8000-8004 also can be used.

Nucleic acids of the present invention can be transferred to cells according to the methods of the present invention, as well as using any suitable means known in the art. The transformed cells can be induced to form transformed plants via organogenesis or embryogenesis, according to the procedures of Dixon (1987) Plant Cell Culture: A Practical Approach (IRL Press, Oxford).

The invention comprises mini-retrotransposons that are modified into a dual vector system. All retrotransposons comprise the same basic elements; this invention relates to LTR retrotransposons which comprise long terminal repeats with autonomous elements containing at least two genes, gag, which encodes a capsid like protein and pol which encodes a protein that has protease, reverse transcriptase, RNase H and integrase activities. Retrotransposon elements useful for the invention include but are not limited to LTR transposons, Tnt1, Tto1 and Tto2 from tobacco, and Tos17 from rice. The sequences of these elements and the basic components that make them up are readily available to those of skill in the art through the references disclosed herein and through sources such as Genbank. Use of a term such as LTR, or retrotransposon element shall include the region defined in any one of these references as well as all variants (conservatively modified or otherwise) which retain biological activity sufficient to function as an active portion of a retrotransposon. Assays for retrotransposon activity are disclosed herein. Retrotransposons and the elements which define them are discussed in the following United States patents which are incorporated herein, U.S. Pat. Nos. 6,027,722; 5,879,933; 5,976,795; 5,354,674 and 6,228,647 as well as references disclosed herein and in Genbank.

Transgenic Techniques Overview

According to the present invention, nucleotide sequences are expressed in transformed plants. Production of genetically modified plant tissue either expressing or inhibiting expression of a nucleotide sequence combines the teachings of the present disclosure with a variety of techniques and expedients known in the art. In most instances, alternate expedients exist for each stage of the overall process. The choice of expedients depends on the variables such as the plasmid vector system chosen for the cloning and introduction of the recombinant DNA molecule, the plant species to be modified, the particular nucleotide sequence ie. structural gene, promoter elements and upstream elements, design of up or down regulation elements, used. Persons skilled in the art are able to select and use appropriate alternatives to achieve functionality. Culture conditions for expressing desired nucleotide sequences and cultured cells are known in the art. Also as known in the art, a number of both monocotyledonous and dicotyledonous plant species are transformable and regenerable such that whole plants containing and expressing desired genes under regulatory control of the promoter molecules according to the invention may be obtained. As is known to those of skill in the art, expression in transformed plants may be tissue specific and/or specific to certain developmental stages. Truncated promoter selection and structural gene selection are other parameters which may be optimized to achieve desired plant expression or inhibition as is known to those of skill in the art and taught herein.

The following is a non-limiting general overview of Molecular biology techniques which may be used in performing the methods of the invention.

Structural Gene

In one embodiment, the nucleotide sequence may be a structural gene, the function of which is desired to be known in a particular plant, or tissue type. Thus by means of the present invention, agronomic genes can be expressed in transformed plants to identify function of the same, temporally or spatially or with a certain promoter combination. Examples of structural genes, the function of which in plant cells may be assayed include:

Plant disease resistance genes, (Martin et al., Science 262: 1432 (1993) (tomato Pto gene for resistance to Pseudomonas syringae pv. tomato encodes a protein kinase)); a Bacillus thuringiensis protein, (Geiser et al., Gene 48: 109 (1986); a lectin, (Van Damme et al., Plant Molec. Biol. 24: 25 (1994)); a vitamin-binding protein, (such as avidin. see PCT application US93/06487); an enzyme inhibitor, (Abe et al., J. Biol. Chem. 262: 16793 (1987)); an insect-specific hormone or pheromone, (see, for example, Hammock et al., Nature 344: 458 (1990)); an insect-specific peptide or neuropeptide, (Regan, J. Biol. Chem. 269: 9 (1994)); an insect-specific venom, (Pang et al., Gene 116: 165 (1992); an enzyme responsible for an hyperaccumulation of a monterpene; an enzyme involved in the modification, including the post-translational modification, of a biologically active molecule; for example, a glycolytic enzyme, a proteolytic enzyme; (See PCT application WO 93/02197); a molecule that stimulates signal transduction, (for example, Botella et al., Plant Molec. Biol. 24: 757 (1994)); a hydrophobic moment peptide, (PCT application WO 95/16776); a membrane permease, (Jaynes et al., Plant Sci. 89: 43 (1993)); a viral-invasive protein or a complex toxin derived therefrom, (Beachy et al., Ann. Rev. Phytopathol.28: 451 (1990)); (Taylor et al., Abstract #497, SEVENTH INT'L SYMPOSIUM ON MOLECULAR PLANT-MICROBE INTERACTIONS (Edinburgh, Scotland, 1994)); a virus-specific antibody, (Tavladoraki et al., Nature 366: 469 (1993)); a developmental-arrestive protein produced in nature by a pathogen or a parasite, (Lamb et al., Bio/Technology 10: 1436 (1992)); a developmental-arrestive protein produced in nature by a plant, (Logemann et al., Bio/Technology 10: 305 (1992)); a herbicide that inhibits the growing point or meristem, such as an imidazalinone or a sulfonylurea, (Lee et al.,EMBO J. 7: 1241 (1988)); Glyphosate (resistance imparted by mutant 5-enolpyruvl-3-phosphikimate synthase (EPSP) and aroA genes, respectively) (U.S. Pat. No. 4,940,835); a herbicide that inhibits photosynthesis, such as a triazine (psbA and gs+genes) and a benzonitrile (nitrilase gene). (Przibilla et al., Plant Cell 3: 169 (1991)); Modified fatty acid metabolism, for example, by transforming a plant with an antisense gene of stearoyl-ACP desaturase to increase stearic acid content of the plant. See Knultzon et al., Proc. Natl. Acad. Sci. USA 89: 2624 (1992); decreased phytate content, (Van Hartingsveldt et al., Gene 127: 87 (1993)); modified carbohydrate composition, for example, by transforming plants with a gene coding for an enzyme that alters the branching pattern of starch. (See Shiroza et al., J. Bacteriol. 170: 810 (1988)); genes that controls cell proliferation and growth of the embryo and/or endosperm such as cell cycle regulators (Bogre L et al., “Regulation of cell division and the cytoskeleton by mitogen-activated protein kinases in higher plants.” Results Probl Cell Differ 27:95-117 (2000).

Promotors

The promoters disclosed herein may be used in conjunction with naturally occurring flanking coding or transcribed sequences of the desired structural gene/s or with any other coding or transcribed sequence that is critical to structural gene formation and/or function.

It may also be desirable to include some intron sequences in the promoter constructs since the inclusion of intron sequences in the coding region may result in enhanced expression and specificity. Thus, it may be advantageous to join the DNA sequences to be expressed to a promoter sequence that contains the first intron and exon sequences of a polypeptide which is unique to cells/tissues of a plant critical to seed specific Structural formation and/or function.

Additionally, regions of one promoter may be joined to regions from a different promoter in order to obtain the desired promoter activity resulting in a chimeric promoter. Synthetic promoters which regulate gene expression may also be used.

The expression system may be further optimized by employing supplemental elements such as transcription terminators and/or enhancer elements.

Other Regulatory Elements

In addition to a promoter sequence, an expression cassette or construct should also contain a transcription termination region downstream of the structural gene to provide for efficient termination. The termination region or polyadenylation signal may be obtained from the same gene as the promoter sequence or may be obtained from different genes. Polyadenylation sequences include, but are not limited to the Agrobacterium octopine synthase signal (Gielen et al., EMBO J. (1984) 3:835-846) or the nopaline synthase signal (Depicker et al., Mol. and Appl. Genet. (1982) 1:561-573).

Marker Genes

Recombinant DNA molecules containing any of the DNA sequences and promoters described herein may additionally contain selection marker genes which encode a selection gene product which confer on a plant cell resistance to a chemical agent or physiological stress, or confers a distinguishable phenotypic characteristic to the cells such that plant cells transformed with the recombinant DNA molecule may be easily selected using a selective agent. One such selection marker gene is neomycin phosphotransferase (NPT II) which confers resistance to kanamycin and the antibiotic G-418. Cells transformed with this selection marker gene may be selected for by assaying for the presence in vitro of phosphorylation of kanamycin using techniques described in the literature or by testing for the presence of the mRNA coding for the NPT II gene by Northern blot analysis in RNA from the tissue of the transformed plant. Polymerase chain reactions are also used to identify the presence of a transgene or expression using reverse transcriptase PCR amplification to monitor expression and PCR on genomic DNA. Other commonly used selection markers include the ampicillin resistance gene, the tetracycline resistance and the hygromycin resistance gene. Transformed plant cells thus selected can be induced to differentiate into plant structures which will eventually yield whole plants. It is to be understood that a selection marker gene may also be native to a plant.

Tranformation

A recombinant DNA molecule whether designed to inhibit expression or to provide for expression containing any of the DNA sequences and/or promoters described herein may be integrated into the genome of a plant by first introducing a recombinant DNA molecule into a plant cell by any one of a variety of known methods. Preferably the recombinant DNA molecule(s) are inserted into a suitable vector and the vector is used to introduce the recombinant DNA molecule into a plant cell.

The use of Cauliflower Mosaic Virus (CaMV) (Howell, S. H., et al, 1980, Science, 208:1265) and gemini viruses (Goodman, R. M., 1981, J. Gen Virol. 54:9) as vectors has been suggested but by far the greatest reported successes have been with Agrobacteria sp. (Horsch, R. B., et al, 1985, Science 227:1229-1231).

Methods for the use of Agrobacterium based transformation systems have now been described for many different species. Generally strains of bacteria are used that harbor modified versions of the naturally occurring Ti plasmid such that DNA is transferred to the host plant without the subsequent formation of tumors. These methods involve the insertion within the borders of the Ti plasmid the DNA to be inserted into the plant genome linked to a selection marker gene to facilitate selection of transformed cells. Bacteria and plant tissues are cultured together to allow transfer of foreign DNA into plant cells then transformed plants are regenerated on selection media. Any number of different organs and tissues can serve as targets from Agrobacterium mediated transformation as described specifically for members of the Brassicaceae. These include thin cell layers (Charest, P. J., et al, 1988, Theor. Appl. Genet. 75:438-444), hypocotyls (DeBlock, M., et al, 1989, Plant Physiol. 91:694-701), leaf discs (Feldman, K. A., and Marks, M. D., 1986, Plant Sci. 47:63-69), stems (Fry J., et al, 1987, Plant Cell Repts. 6:321-325), cotyledons (Moloney M. M., et al, 1989, Plant Cell Repts. 8:238-242) and embryoids (Neuhaus, G., et al, 1987, Theor. Appl. Genet. 75:30-36), or even whole plants using in vacuum infiltration and floral dip or floral spraying transformation procedures available in Arabidopsis and Medicago at present but likely applicable to other plants in the hear future. It is understood, however, that it may be desirable in some crops to choose a different tissue or method of transformation.

Other methods that have been employed for introducing recombinant molecules into plant cells involve mechanical means such as direct DNA uptake, liposomes, electroporation (Guerche, P. et al, 1987, Plant Science 52:111-116) and micro-injection (Neuhaus, G., et al, 1987, Theor. Appl. Genet. 75:30-36). The possibility of using microprojectiles and a gun or other device to force small metal particles coated with DNA into cells has also received considerable attention (Klein, T. M. et al., 1987, Nature 327:70-73).

In accordance with the invention, it is not necessary for the vector to be expressed or integrated to reproductive cells of the plant.

The regenerated plants are transferred to standard soil conditions and cultivated in a conventional manner.

Following transformation of target tissues, expression of the above-described selectable marker genes allows for preferential selection of transformed cells, tissues and/or plants, using regeneration and selection methods now well known in the art.

The foregoing methods for transformation would typically be used for producing a transgenic variety. The transgenic variety could then be crossed, with another (non-transformed or transformed) variety, in order to produce a new transgenic variety. Alternatively, a genetic trait which has been engineered into a particular maize line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties which do not contain that gene. As used herein, “crossing” can refer to a simple X by Y cross, or the process of backcrossing, depending on the context.

The following examples serve to better illustrate the invention described herein and are not intended to limit the invention in any way. All references cited herein are hereby expressly incorporated to this document in their entirety by reference.

### EXAMPLES

Generating the Tnt1 Element

The functional Tnt1 retrotransposon was constructed using a PCR based strategy. The entire element was assembled from four different parts that were PCR amplified from tobacco genomic DNA, cloned and sequenced. Due to degeneracy of the element in the genome, sequence mismatches at a frequency of 0.5% (in comparison to X13777) were observed. Nucleotide mismatches were repaired using overlapping primers and resulting clones were then used to assemble a full-length element that matched the published sequence. Transcription of the Tnt1 element in tobacco is induced by microbial elicitors, pathogens and abiotic stresses such as wounding and freezing (Mhiri et al., 1997; Melayah et al., 2001). Additionally, constitutive expression of Tnt1 in a heterologous host has been obtained by using the 35S CaMV promoter to drive transcription (Lucas et al., 1995; Hirochika et al., 1996). A second Tnt1 clone was created through a PCR based method using overlapping primer pairs to fuse the 35S promoter to the transcriptional start site of the Tnt1 element. Both retrotransposons were tested for transposition competence and found to be active.

Construction of the Mini-Tnt1 Vector and Helper Elements

We next developed Tnt1 clones as vector retroelements to carry exogenous DNA and helper retroelements to supply Gag and Pol proteins in trans. The vector retroelements were designed so as to carry only the cis acting sequences necessary to allow reverse transcription. Two different versions of the vector retroelements were generated. Transcription from one version was under the control of the native LTR (expression of which will be induced under stress; SEQ ID NO: 1 and FIG. 1A;) while the other was under the control of the CaMV 35S promoter (constitutive expression of the mini-element; SEQ ID NO: 2 and FIG. 1B). A polylinker with unique restriction sites was added to each of the vector retroelements to facilitate cloning of exogenous DNA (FIGS. 1A and 1B). To provide a marker gene to follow transposition, the coding sequence of NPT II was modified so that it was disrupted by an intron and then placed under the transcriptional control of the nopaline synthase (NOS) promoter. This marker was inserted into the vector retroelements such that the Tnt1 3′ LTR acted as a transcriptional terminator for both the vector retroelement and the marker gene (SEQ ID NO: 3).

The Tnt1 helper retroelement was constructed by placing the gag and pol coding sequences under control of the CaMV 35S promoter and NOS transcriptional terminator (SEQ ID NO: 8)

Protoplast Transformation and Generation of Stably Transformed Calli

Nicotiana tobaccum cv. Xanthi was used to make tobacco protoplasts. Expanded leaf tissue from in vitro grown plants was digested using a protocol designed by Fromm et al. (1987) with appropriate modification. Selection for NPT II and regeneration of calli from protoplasts was carried out using established protocols (Van den Elzen et al., 1985). To obtain protoplasts from Arabidopsis thaliana, enzymatic digestion of leaves was performed using adaptations from Fromm et al. (1987). Arabidopsis regeneration was carried out based on procedures from Wenck and Marton (1995). Additional transformations of Arabidopsis was performed through the Agrobacterium-mediated floral dip method (Desfeux et. al., 2000).

Assay for cDNA Synthesis

To assay for transposition of the Tnt1 vector retroelements, advantage was taken of the intron in the NPT II gene cloned within the Tnt1 vector retroelement SEQ ID NO:3. The NPT II gene carries an intron, which is spliced from the mRNA so that reverse transcription creates an intronless cDNA. This enables the parental and progeny element to be distinguished by PCR (FIG. 2), i. e. cDNA is about 80 bp shorter than the parental construct. For the PCR reactions, one primer resides within the Tnt1 sequence (Primer1: DVO1667 5′-TGAAAAATAAAAATGTCTGGAGTAAAGTACGAGGTA-3′SEQ ID NO:4) and the other resides in the NPT II gene (Primer 2: DVO1107-5′ CCTTCCCGCTTCAGTGACAACGTCGA3′SEQ ID NO:5). To conduct the assay, tobacco protoplasts were electroporated with the vector retroelements carrying the intron-disrupted NPT II gene. Tnt1 Gag and Pol polyproteins were provided in trans as a result of activation of the native Tnt1 element during protoplast formation (Pouteau et al. 1991). Individual cells were grown on callus inducing media containing kanamycin. Genomic DNA was isolated from resistant calli and was analyzed for the presence or absence of the intron. Three of eighteen calli analyzed had an amplified product corresponding to the size expected due to a splicing event (FIG. 2). Sequence analysis of the bands indicated that the intron was spliced out thereby suggesting transposition of the Tnt1 vector retroelement had occurred

We tested the ability of the Tnt1 vector retroelements to transpose in the heterologous host Arabidopsis. thaliana. Arabidopsis protoplasts were electroporated with the 35S-driven vector retroelement carrying the modified NPT II gene. The Gag-Pol polyprotein was provided in trans during electroporation. Kanamycin resistant calli containing newly transposed Tnt1 vectors are expected to have the 35S promoter replaced by the 5′LTR. Genomic DNA from kanamycin resistant calli was amplified using a primer present in the 5′ LTR (PDIO-261 5′-CATTGAAGAAGTATTAGGCATGT-3′ SEQ ID NO:6) and a reverse primer (DVO1819 5′-TCCTCAGCTTTCATGGTATCAGGC-3′ SEQ ID NO:7), which lies in the gag coding region. Restoration of the 5′ end of the LTR was observed in three of twenty calli analyzed (FIG. 3B). Sequencing of these bands indicated reconstitution of the 5′ LTR sequence.

All references cited herein are hereby expressly incorporated herein in their entirety by reference, including but not limited to the following:
Courtial B., Feuerbach F., Eberhard S., Rohmer L., Chiapello H., Camilleri C., and Lucas H. 2001. Tnt1 transposition events are induced by in vitro transformation of Arabidopsis thaliana, and transposed copies integrate into genes. Molecular Genetics and Genomics 265: 32-42
Coffin, J. M., Hughes, S. H., and Varmus, H. 1997. Retroviruses. Cold Spring Harbor Laboratory Press, Cold Spring Harbor, N.Y.
Desfeux C., Clough S. J., and Bent A. F. 2000. Female reproductive tissues are the primary target of Agrobacterium-mediated transformation by the Arabidopsis floral-dip method. Plant Physiology. Vol. 123 895-904.
Feuerbach F., Drouaud J., and Lucas H 1997. Retrovirus-like end processing of the Tobacco Tnt1 retrotransposon linear intermediates of replication. J. Virology. May 4005-4015.
Fromm, M., Callis, J., Taylor, L. P. and Walbot, V. (1987) Electroporation of DNA and RNA into plant protoplasts. Methods Enzymol., 153, 351-366.
Hirochika H. 1993. Activation of tobacco retrotransposon during tissue culture EMBO Journal 12(6) 2521-2528.
Hirochika H. and Otsuki H. 1995. Extrachromosomal circular forms of the tobacco retrotransposon Tto1. Gene 165: 229-232.
Hirochika H., Otsuki H., Yoshikawa M., Otsuki Y., Sugimoto K. and Takeda S. 1996. Autonomous transposition of the tobacco transposon Tto1 in rice. The Plant Cell 8 725-734.
Hirochika H, Guiderdoni E, An G, Hsing Y I, Eun M Y, Han C D, Upadhyaya N, Ramachandran S, Zhang Q, Pereira A, Sundaresan V, Leung H. 2004 Rice mutant resources for gene discovery. Plant Mol Biol. 54:325-34.
Isabelle d'Erfurth., Cosson. V., Eschstruth A., Lucas H., Kondorosi A. and Ratet P. 2003. Efficient transposition of the Tnt-1 tobacco retrotransposon in the model legume Medicago trunculata. Plant J. 34:95-106.
Jaaskelainen M., Mykkanen A. H., Arna T., Vicient C. M., Suoniemi A., Kalender R., Savilahti H. and Schulman A. H. 1999. Retrotransposon BARE-1: expression of encoded proteins and formation of virus-like particles in barley cells. Plant J. 20(4) 413-422.
Ke, N., and Voytas D. F. 1997. High frequency cDNA recombination of the Saccharomyces retrotransposon Ty5: The LTR mediates formation of tandem elements. Genetics 147:545-56.
Ke, N., and Voytas D. F. 1999. cDNA of the Yeast retrotransposon Ty5 Preferentially Recombines with substrates in Silent Chromatin. Mol. Cell. Biol. 19:484494.
Lucas H., Feuerbach F., Kunert K., Grandbastein M. A. and Caboche M. 1995 RNA mediated transposition of the tobacco retrotransposon Tnt1 in Arabidopsis thaliana. The EMBO J. Vol. 14 (10) 2364-2373.
Maniatis T., Fritsch E. F., Sambrook J. 1989. Molecular cloning. A laborartory manual. (Cold Spring Harbor, Cold Spring Harbor Laborartory Press)
Melayah D., Bonnivard E., Chalhoub B., Audeon C. and Grandbastein M A. 2001. The mobility of the tobacco Tnt1 retrotransposon correlates with its transcriptional activation by fungal factors. The Plant J. 28(2) 159-168.
Mhiri C., Morel J B., Vernhettes S., Casacuberta J M., Lucas H. and Granbastein M A. 1997. The promoter of the Tnt1 retrotransposon is induced by wounding and abiotic stress. Plant Mol. Biol. 33:257-266.
Miyao A, Tanaka K, Murata K, Sawaki H, Takeda S, Abe K, Shinozuka Y, Onosato K, Hirochika H. 2003. Target site specificity of the Tos17 retrotransposon shows a preference for insertion within genes and against insertion in retrotransposon-rich regions of the genome. Plant Cell. 15:1771-80.
Pouteau S., Huttner E., Granbastein M A. and Caboche M. 1991. Specific expression of the tobacco Tnt1 retrotransposon in protoplasts EMBO J. 10: 1911-1918.
Sundaresan V. 1996. Horizontal spread of transposon mutagenesis: new uses for old elements. Trends Plant Sci. 1:184-190.
Van den Elzen P. J. M., Townsend J., Lee K. Y., and Bedbrook J. R. 1985. A chimeric hygromycin resistance gene as a selectable marker in plant cells. Plant Mol. Biol. 5:149-154.
Wenck A. R., and Marton L 1995. Large scale protoplast isolation and regeneration of Arabidopsis thaliana. Biotechniques. Vol.18 No.4 640-643.

### Example 2

The following is annotated sequence information for the present invention:

Seq ID NO:1 pIP62 Wild type mini-Tnt1 2034 bp: 1-6: Unique ClaI cloning site for removal of the entire mini-Tnt1 cassette 7-616: Tnt1 5′ LTR 617-697: non-coding leader sequence 698-1012: Translation start and Tnt1 Gag coding sequence 1013-1095: Multiple cloning sites 1096-1372: C-terminus of Tnt1 Pol up to the stop codon 1373-1418: Tnt1 non-coding sequence between stop codon and 3′ LTR 1419-2028: Tnt1 3′ LTR 2029-2034: Unique ApaI Cloning site for removal of the entire mini-Tnt1 cassette

Seq ID NO:2 pIP65 35S mini-Tnt1 2681 bp: 1-56: Linker sequence with unique ClaI and BsiWI sites for removal of the entire mini-Tnt1 cassette 57-886: 35S promoter region 887-1263: Tnt1 5′ LTR 1264-1344: non coding leader sequence 1345-1659: Translation start and Tnt1 Gag coding sequence 1660-1742: Multiple cloning sites 1743-2019: C-terminus of Tnt1 Pol up to the stop codon 2020-2065: Tnt1 non-coding sequence between stop codon and 3′ LTR 2066-2675: Tnt1 3′ LTR 2676-2681: Unique ApaI Cloning site for removal of the entire mini-Tnt1 cassette

Seq ID NO:3 35S Mini Tnt1 with nptII gene 1-12: Linker sequence 12-842: 35S promoter 843-1219: Partial sequence of Tnt1 LTR; position 843 is the Tnt1 transcriptional start site 1220-1300: non-coding leader sequence 1301-1615: Translation start and Tnt1 Gag coding sequence 1616-1621: XhoI site (in lower case) 1622-2825: Nos promoter and the nptII gene with the intron (lower case) 2826-2831: Nde1 site (lower case) 2832-3155: C-terminus of Tnt1 Pol and non-coding sequence between stop codon and 3′ LTR 3156-3765: 3′ Tnt1 LTR 3766-3771: ApaI cloning site

Seq ID NO:8 35S Tnt1 gag/pol and Nos terminator 1-6: claI cloning site 7-850: 35S promoter 860-4873: Tnt1 gag/pol coding region with modifications to include XhoI and SacII cloning sites at the 5′ and 3′ ends respectively 4874-5137: Nos terminator 5138-5143: ClaI cloning site

1. A mini-retrotransposon vector comprising the following plant retrotransposon elements:
a 5′ long terminal repeat sequence; a promoter sequence operatively linked to said 5′ long terminal repeat sequence, a 3′ long terminal repeat sequence; and a polypurine tract.

2. The vector of claim 1 further comprising: a primer binding site.

3. The vector of claim 1 further comprising a polylinker with unique restriction sites to facilitate cloning of exogenous DNA.

4. The vector of claim 1 further comprising a portion of gag.

5. The vector of claim 1 wherein said promoter is a constitutive promoter.

6. The vector of claim 1 wherein said promoter is an inducible promoter.

7. The vector of claim 5 wherein said promoter is a 35S promoter.

8. The vector of claim 1 wherein said retrotransposon elements are selected from the group consisting of Tnt1, Tto1, and Tos17.

9. The vector of claim 1 wherein said retrotransposon elements are from Tnt1.

10. The vector of claim 1 wherein said vector comprises SEQ ID NO:2.

11. The vector of claim 1 further comprising a heterologous nucleotide sequence the expression of which is desired in a plant cell.

12. A host plant cell comprising the vector of claim 1.

13. A mini-retrotransposon vector comprising the following Tnt1 plant retrotransposon elements:
a 5′ long terminal repeat sequence; a 3′ long terminal repeat sequence; and a polypurine tract.

14. The vector of claim 13 further comprising: a primer binding site.

15. The vector of claim 13 further comprising a polylinker with unique restriction sites to facilitate cloning of exogenous DNA.

16. The vector of claim 13 further comprising a portion of gag.

17. The vector of claim 13 wherein said promoter is a constitutive promoter.

18. The vector of claim 13 wherein said vector comprises a sequence selected from SEQ ID NO:1 or SEQ ID NO:2.

19. The vector of claim 13 further comprising a heterologous nucleotide sequence the expression of which is desired in a plant cell.

20. A plant cell comprising the vector of claim 13.

21. A method of transforming a plant cell comprising:
introducing to said plant cell a vector comprising retrotransposon elements of a 5′ long terminal repeat sequence; a promoter sequence operatively linked to said 5′ long terminal repeat sequence, a 3′ long terminal repeat sequence; a polypurine tract and a nucleotide sequence the presence of which is desired in a plant cell, wherein said vector does not include gag and pol genes, wherein said introduction is in the presence of gag and pol genes of a retrotransposon so that an active retroelement is formed.

22. The method of claim 21 wherein said gag and pol elements are provided by endogenous host retrotransposon elements.

23. The method of claim 21 wherein said gag and pol elements are provided by a second helper vector.

24. The method of claim 21 wherein said gag and pol elements are provided by a host cell which has been transformed with heterologous gag and pol sequences.

25. The method of claim 21 wherein said vector elements are from a Tnt 1 retrotranspsoson.

26. A plant cell which includes heterogenous polynucleotides produced by the method of claim 21.

27. A method of transforming a plant cell comprising:
introducing to said plant cell a vector comprising Tnt1 retrotransposon elements of a 5′ long terminal repeat sequence; a 3′ long terminal repeat sequence; a polypurine tract and a nucleotide sequence the presence of which is desired in a plant cell, wherein said vector does not include gag and pol genes, wherein said introduction is in the presence of gag and pol retrotransposon elements so that and active retroelement is formed.

28. The method of claim 27 wherein said gag and pol elements are provided by endogenous host retrotransposon elements.

29. The method of claim 27 wherein said gag and pol elements are provided by a second helper vector.

30. The method of claim 27 wherein said gag and pol elements are provided by a host cell which has been transformed with heterologous gag and pol sequences.

31. The method of claim 27 wherein said vector further comprises a constitutive promoter operably linked to said 5′ long terminal repeat.

32. A plant cell which includes heterogenous polynucleotides produced by the method of claim 27.


SEQUENCE LISTING (8 sequences)

SEQ ID NO: 1 - DNA, 2034 bp, nicotania
atcgattgatgatgtccatctcattgaagaagtattaggcatgtgcctaataagagttttctttggtttggtagccaaccttgttgacttggtttggttggtagccaaccttgttgaatccttgttggattggtagccaactttgttgaattgtgaaaaatgtgtgtaaattgtcaaatattgtaggctttagagggtgaagctttggctataaaaggagagcttcaactctcatttcttcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacaactggtatcagagcacaggttctgctcgttcactgaaatactattcactgtcggtagtactatacttggtgaaaaataaaaatgtctggagtaaagtacgaggtagcaaaattcaatggagataacggtttctcaacatggcaaagaaggatgagagatctgctcatccaacaaggattacacaaggttctagatgttgattccaaaaagcctgataccatgaaagctgaggattgggctgacttggatgaaagagctgctagtgcaatcaggttgcacttatcagatgatgtggtaaataacatcattgatgaagacactgcacgtggaatttggacaaggttggaaagcctatacatgtccaaaacgctgacaaataaattgtacctgaagaagctcgagcgtacggcgccggatccgaattctccccgcggaggtacctcccccgggatgagctctactagtaaacgcgtcatatgcaagcgattccttcaagagcttggattgcatcagaaggagtatgtcgtctattgtgacagtcaaagtgcaatagaccttagcaagaactctatgtaccatgcaaggaccaaacacattgatgtgagatatcattggattcgagaaatggtagatgatgaatctctaaaagtcttgaagatttctacaaatgagaatcccgcagatatgctgaccaaggtggtaccaaggaacaagttcgagctatgcaaagaacttgtcggcatgcattcaaactagaagacagtgctacctcctctggatgaatgagactggagggggagattgatgatgtccatctcattgaagaagtattaggcatgtgcctaataagagttttctttggtttggtagccaaccttgttgacttggtttggttggtagccaaccttgttgaatccttgttggattggtagccaactttgttgaattgtgaaaaatgtgtgtaaattgtcaaatattgtaggctttagagggtgaagctttggctataaaaggagagcttcaactctcatttcttcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacagggccc

SEQ ID NO: 2 - DNA, 2681 bp, nicotania
atcgatggcgccagctgcaggaattcgatatcaagcttatcgatcgtacggtccccagatttgccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagatcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacaactggtatcagagcacaggttctgctcgttcactgaaatactattcactgtcggtagtactatacttggtgaaaaataaaaatgtctggagtaaagtacgaggtagcaaaattcaatggagataacggtttctcaacatggcaaagaaggatgagagatctgctcatccaacaaggattacacaaggttctagatgttgattccaaaaagcctgataccatgaaagctgaggattgggctgacttggatgaaagagctgctagtgcaatcaggttgcacttatcagatgatgtggtaaataacatcattgatgaagacactgcacgtggaatttggacaaggttggaaagcctatacatgtccaaaacgctgacaaataaattgtacctgaagaagctcgagcgtacggcgccggatccgaattctccccgcggaggtacctcccccgggatgagctctactagtaaacgcgtcatatgcaagcgattccttcaagagcttggattgcatcagaaggagtatgtcgtctattgtgacagtcaaagtgcaatagaccttagcaagaactctatgtaccatgcaaggaccaaacacattgatgtgagatatcattggattcgagaaatggtagatgatgaatctctaaaagtcttgaagatttctacaaatgagaatcccgcagatatgctgaccaaggtggtaccaaggaacaagttcgagctatgcaaagaacttgtcggcatgcattcaaactagaagacagtgctacctcctctggatgaatgagactggagggggagattgatgatgtccatctcattgaagaagtattaggcatgtgcctaataagagttttctttggtttggtagccaaccttgttgacttggtttggttggtagccaaccttgttgaatccttgttggattggtagccaactttgttgaattgtgaaaaatgtgtgtaaattgtcaaatattgtaggctttagagggtgaagctttggctataaaaggagagcttcaactctcatttcttcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacagggccc

SEQ ID NO: 3 - DNA, 3771 bp, Nicotania
cgtacggtccccagatttgccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagatcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacaactggtatcagagcacaggttctgctcgttcactgaaatactattcactgtcggtagtactatacttggtgaaaaataaaaatgtctggagtaaagtacgaggtagcaaaattcaatggagataacggtttctcaacatggcaaagaaggatgagagatctgctcatccaacaaggattacacaaggttctagatgttgattccaaaaagcctgataccatgaaagctgaggattgggctgacttggatgaaagagctgctagtgcaatcaggttgcacttatcagatgatgtggtaaataacatcattgatgaagacactgcacgtggaatttggacaaggttggaaagcctatacatgtccaaaacgctgacaaataaattgtacctgaagaagctcgagggtaccggatcatgagcggagaattaagggagtcacgttatgacccccgccgatgacgcgggacaagccgttttacgtttggaactgacagaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatctgatcatgtggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtcaggtaagtttatcagttaaatataataaataaagaagaaaaccaaaaaaatggctaactaaaacgatggtcttatgattttatgcaggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttccatatgtcaagcgattccttcaagagcttggattgcatcagaaggagtatgtcgtctattgtgacagtcaaagtgcaatagaccttagcaagaactctatgtaccatgcaaggaccaaacacattgatgtgagatatcattggattcgagaaatggtagatgatgaatctctaaaagtcttgaagatttctacaaatgagaatcccgcagatatgctgaccaaggtggtaccaaggaacaagttcgagctatgcaaagaacttgtcggcatgcattcaaactagaagacagtgctacctcctctggatgaatgagactggagggggagattgatgatgtccatctcattgaagaagtattaggcatgtgcctaataagagttttctttggtttggtagccaaccttgttgacttggtttggttggtagccaaccttgttgaatccttgttggattggtagccaactttgttgaattgtgaaaaatgtgtgtaaattgtcaaatattgtaggctttagagggtgaagctttggctataaaaggagagcttcaactctcatttcttcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacagggccc

SEQ ID NO: 4 - DNA, 36 bp, Nicotania
tgaaaaataaaaatgtctggagtaaagtacgaggta

SEQ ID NO: 5 - DNA, 26 bp, Nicotania
ccttcccgcttcagtgacaacgtcga

SEQ ID NO: 6 - DNA, 23 bp, Nicotania
cattgaagaagtattaggcatgt

SEQ ID NO: 7 - DNA, 24 bp, Nicotania
tcctcagctttcatggtatcaggc

SEQ ID NO: 8 - DNA, 5143 bp, Nicotania
atcgatgtccccagatttgccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagactcgagaaggagatataacaatgtctggagtaaagtacgaggtagcaaaattcaatggagataacggtttctcaacatggcaaagaaggatgagagatctgctcatccaacaaggattacacaaggttctagatgttgattccaaaaagcctgataccatgaaagctgaggattgggctgacttggatgaaagagctgctagtgcaatcaggttgcacttatcagatgatgtggtaaataacatcattgatgaagacactgcacgtggaatttggacaaggttggaaagcctatacatgtccaaaacgctgacaaataaattgtacctgaagaagcagttatacgccctacacatgagtgaaggtacgaattttttgtcacatttaaatgtgtttaacggactaatcacacagcttgccaacctcggagtgaaaatcgaggaagaagataaagccatcttgctattgaactcgttgccatcttcgtacgataatttggcaacaaccatcctgcacggtaagactactattgagttgaaagatgtcacatcggctcttctactcaatgagaagatgagaaagaagcctgaaaatcaaggacaggctctcatcacagaaggtagaggcaggagttatcaaaggagttcgaacaactatggtagatccggagctcgtgggaagtcaaagaatcgatccaaatcaagagtcagaaattgctacaactgtaatcaaccaggtcacttcaaaagagattgcccaaatccaaggaagggcaaaggtgaaaccagtggccagaagaatgacgacaacacagccgccatggtgcaaaataatgataatgttgtcctctttataaatgaggaagaggaatgcatgcacctgtcaggtccagagtcggaatgggtggttgacacagcggcatctcaccatgccacaccggtaagagatcttttttgcagatatgtagcaggtgatttcggcacagtgaaaatgggtaacacaagttactcaaagattgcggggattggtgacatttgtatcaagacaaatgtcggatgcacattggttctaaaggatgtgcggcatgtacctgatttgcggatgaacttgatctcgggaattgctttagaccgagatggatacgagagttattttgcaaatcaaaagtggagactcactaagggatcattggtgattgcaaagggagttgctcgtggcacgttgtacaggacaaatgcagaaatatgccaaggtgaattgaacgcggcacaagatgagatttctgtagatttatggcacaaaagaatgggtcatatgagcgagaagggattgcagattcttgccaagaaatcactcatttcttatgccaaaggtacaactgtaaaaccttgtgactactgtttatttggtaagcagcatagagtctcatttcagacatcgtctgaaagaaaattgaatatacttgatttagtatattctgatgtttgcggcccaatggaaattgaatcaatgggcggtaacaaatattttgttacttttattgatgatgcttcacgaaaattatgggtttatattttgaaaaccaaagatcaggtgtttcaagttttccagaagtttcatgctctagtagaaagggagacgggtcgaaagctaaagcgtctccgaagtgacaatggaggtgagtacacttcaagggaatttgaagagtattgttcaagtcatgggatcagacatgaaaagacagttcctggaaccccacagcacaatggcgtagccgagaggatgaaccgcaccattgtggagaaggtgagaagcatgctcagaatggctaaactgcctaagtcattctggggtgaagcagttcagacagcctgttacctgatcaataggagtccatcagttccgttggcgtttgaaatcccagagagagtctggaccaacaaggaggtgtcctactcgcatctgaaggtgttcggttgcagagcttttgcacatgtaccaaaagagcagagaacaaagctggatgataaatctattccctgcatatttatcggatatggagatgaagagttcgggtacagactgtgggatcctgtaaagaagaaggtcatcagaagtagagatgtagtcttccgagaaagtgaagttagaactgctgctgatatgtcagaaaaggtgaagaatggtataattcctaactttgttactattccttctacttctaacaatcccacaagtgcagaaagtacgaccgacgaggtttccgagcagggggagcaacctggtgaggttattgagcagggggagcaacttgatgaaggtgtcgaggaagtggagcaccccactcagggagaagaacaacatcaacctctgaggagatcagagaggccaagggtagagtcacgcaggtacccttccacagagtatgtcctcatcagtgatgatagggagccagaaagtcttaaggaggtgttgtcccatccagaaaagaaccagttgatgaaagctatgcaagaagagatggaatctctccagaaaaatggcacatacaagctggttgaacttccaaagggtaaaagaccactcaaatgcaaatgggtctttaaactcaagaaagatggagattgcaagctggtcagatacaaagctcgattggtggttaaaggcttcgaacagaagaaaggtattgattttgacgaaattttctcccccgttgttaaaatgacttctattcgaacaattttgagcttagcagctagcctagatcttgaagtggagcagttggatgtgaaaactgcatttcttcatggagatttggaagaggagatttatatggagcaaccagaaggatttgaagtagctggaaagaaacacatggtgtgcaaattgaataagagtctttatggattgaagcaggcaccaaggcagtggtacatgaagtttgattcattcatgaaaagtcaaacatacctaaagacctattctgatccatgtgtatacttcaaaagattttctgagaataactttattatattgttgttgtatgtggatgacatgctaattgtaggaaaagacaaggggttgatagcaaagttgaaaggagatctgtccaagtcatttgatatgaaggacttgggcccagcacaacaaattctagggatgaagatagttcgagagagaacaagtagaaagttgtggctatctcaggagaagtacattgaacgtgtactagaacgcttcaacatgaagaatgctaagccagtcagcacacctcttgctggtcatctaaagttgagtaaaaagatgtgtcctacaacagtggaagagaaagggaacatggctaaagttccttattcttcagcagtcggaagcttgatgtatgcaatggtatgtactagacctgatattgctcacgcagttggtgttgtcagcaggtttcttgaaaatcctggaaaggaacattgggaagcagtcaagtggatactcaggtacctgagaggtaccacgggagattgtttgtgctttggaggatctgatccaatcttgaagggctatacagatgctgatatggcaggtgacattgacaacagaaaatccagtactggatatttgtttacattttcagggggagctatatcatggcagtctaagttgcaaaagtgcgttgcactttcaacaactgaagcagagtacattgctgctacagaaactggcaaggagatgatatggctcaagcgattccttcaagagcttggattgcatcagaaggagtatgtcgtctattgtgacagtcaaagtgcaatagaccttagcaagaactctatgtaccatgcaaggaccaaacacattgatgtgagatatcattggattcgagaaatggtagatgatgaatctctaaaagtcttgaagatttctacaaatgagaatcccgcagatatgctgaccaaggtggtaccaaggaacaagttcgagctatgcaaagaacttgtcggcatgcattcaaactagccgcgggaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgtatcgat